(19) United States
(12) Patent Application Publication
(10) Pub. No.: US 2015/0132420 A1
(43) Pub. Date: May 14, 2015

(54) PROCESSES FOR PREPARING A CARBOHYDRATE EXTRACT COMPRISING MANNOHEPTULOSE AND COMPOSITIONS COMPRISING SAME

(71) Applicant: The Iams Company, Mason, OH (US)

(72) Inventors: Maria Dolores Martinez-Serna Villagran, Mason, OH (US); Susan Ruth Beyer, Lebanon, OH (US); Robbert H. Ter Haar, Lebanon, OH (US)

(21) Appl. No.: 14/538,737

(22) Filed: Nov. 11, 2014

Related U.S. Application Data
(60) Provisional application No. 61/902,321, filed on Nov. 11, 2013.
(60) Provisional application No. 61/902,322, filed on Nov. 11, 2013.

(51) Int. Cl. A61K 36/54; A61K 31/047; A61K 31/7004; A23L 1/30; A23K 1/16

(52) U.S. Cl. CPC A61K 36/54; A23L 1/3002; A23K 1/1643; A61K 31/7004; A61K 31/047; A23V 2002/00
USPC 424/769

(57) ABSTRACT

Disclosed herein is a process for preparing a carbohydrate extract comprising mannoheptulose from avocados, as well as compositions, including food compositions, comprising such extracts. The process includes separating the aqueous emulsion into different fractions by centrifugation, and isolating the water-soluble fraction (water extract) comprising avocado carbohydrates, including mannoheptulose. In various aspects, the process optionally includes steps of heating, ultrafiltration, nanofiltration, concentrating, and or drying the extract. Also described herein are methods for using the carbohydrate extract comprising mannoheptulose and compositions comprising the carbohydrate extract comprising mannoheptulose.

### FIELD OF THE DISCLOSURE

The disclosure relates to processes for preparing a carbohydrate extract comprising mannoheptulose and/or perseitol from avocados, as well as compositions comprising such an extract. In certain embodiments, the extracts or compositions prepared thereby are used in preparing food compositions, including pet food compositions.

### BACKGROUND

Mannoheptulose is a seven-carbon sugar, originally identified by LaForge (J. Biol. Chem. 28:511-22, 1917). Mannoheptulose is present in vegetables and fruits, such as avocado, alfalfa, fig, and primrose. The greatest content of mannoheptulose and/perseitol, another seven-carbon sugar, have been reported in avocados. Mannoheptulose and perseitol are unstable in avocados and decline rapidly as the fruit ripens. Interconversion of mannoheptulose and perseitol has been reported by Tesfay et. al; 2010. Perseitol can oxidize to mannoheptulose by enzymes present in the extract of the fruit. This invention describes carbohydrate extracts from avocados that contain particularly high amounts of mannoheptulose and its related seven carbon sugar alcohol perseitol, comprising together about 50-90% of the total soluble sugars. Other six carbon sugars present in the extracts include glucose, fructose, and sucrose.

Mannoheptulose is of interest because it is a classical inhibitor of glucose-induced insulin secretion and glucose oxidation. Mannoheptulose inhibits glucose-induced insulin secretion by selectively inhibiting the enzyme glucokinase. By blocking glucose phosphorylation, the breakdown of glucose is inhibited. Mannoheptulose has also been implicated as an anti-cancer agent, most likely due to its ability to inhibit cell growth in cell types expressing glucokinase, such as in liver tumor cells. Additionally mannoheptulose as been described as an anti-oxidant (Tesfay et al; 2010). As such, mannoheptulose has been implicated as having a variety of effects on mammalian metabolism and health.

Procedures for mannoheptulose extraction from avocados were described previously by LaForge (supra), Kappler-Tanydyaya et al. (Biotechnology J. 2:692-9, 2007), and in U.S. Patent Publication No. 2005/0249837. Some of these procedures took days for extraction and did not provide much mannoheptulose. The present disclosure provides an improved process for obtaining mannoheptulose, resulting in higher yields of mannoheptulose relative to the starting material without compromising the integrity of the mannoheptulose. This improved process may also generate, as by-products, avocado oil and avocado solids that are rich in vitamins A, B, D, E, lutein, carotenoids, and/or proteins useful in health and beauty care formulations.

### SUMMARY OF THE DISCLOSURE

The disclosure is based on the discovery of a new process of preparing a carbohydrate extract comprising mannoheptulose and/or perseitol from avocados using centrifugation. In various aspects, the new process for preparing the extract is quicker and provides higher yields of mannoheptulose and perseitol relative to the starting material without compromising the integrity of the mannoheptulose. Thus, in certain aspects, the advantage of the shorter processing time is that it reduces microbial growth and contamination and reduces risk of degradation of mannoheptulose and/or perseitol.

In one embodiment described herein is a process for preparing a carbohydrate extract comprising mannoheptulose, the process comprising separating an aqueous phase from other phases of an avocado emulsion by centrifugation to provide a carbohydrate extract comprising at least about 2% mannoheptulose. In an alternate embodiment, the present invention provides for a process for preparing a carbohydrate extract comprising mannoheptulose, the process comprising separating water-soluble components from an avocado emulsion by centrifugation to provide a carbohydrate extract comprising at least about 2% mannoheptulose.

In some aspects, the avocado emulsion is formed by grinding avocados in water. In some aspects, the process further comprises heating the water-soluble components. In some aspects, the process further comprises filtering the water-soluble components by ultrafiltration, although such filtering steps are not required. In some aspects, the process further comprises heating the water-soluble components; and subsequently filtering the water-soluble components by ultrafiltration to provide a carbohydrate extract comprising at least about 2% mannoheptulose per wet weight avocado. In another aspect, the process comprises heating the aqueous avocado emulsion prior to centrifugation; separating water-soluble components from the avocado emulsion by centrifugation; and filtering the water-soluble components by ultrafiltration. In some aspects, the ultrafiltration is carried out on a membrane of at least about 10 KDa. In some aspects, centrifugation is carried out after heating.

In some aspects, the process further comprises filtering the water-soluble components by nanofiltration.

In one aspect, the avocado emulsion is emulsified by grinding avocados in water. In one aspect, the water to avocado ratio in the avocado emulsion is at least about 2:1. In another aspect, the water to avocado ratio in the avocado emulsion is at least about 3:1. In another aspect, carbohydrates in the avocado emulsion are solubilized in the aqueous phase of the avocado emulsion by heating. In some aspects, the avocado emulsion is further combined with an enzyme and/or an acid.

In some aspects, the process further comprises drying the carbohydrate extract comprising mannoheptulose. In various aspects, the process further comprises concentrating the carbohydrate extract comprising mannoheptulose utilizing at least one concentration method selected from the group consisting of heating, vacuum drying, evaporating, refractance window drying, freeze drying, and spray drying. In one embodiment, the carbohydrate extract is dried to a point that it is in powder form.

In some aspects, the pH of the avocado emulsion (or aqueous phase of the avocado emulsion after centrifugation) is no great than about 4.0. In some aspects, the process further comprises lowering pH of the water-soluble components to about pH 4.0 or less, where pH values include pH 3.5, pH 3.6, pH 3.7, pH 3.8, pH 3.9 and values there between.

In some aspects, centrifugation is carried out by a horizontal or vertical bowl centrifuge. In some aspects, centrifugation is carried out by a two-phase separator or a three-phase separator. In some aspects, the three-phase separator is a tricanter.

In some aspects, centrifugation is carried out with a relative centrifugal force (G force) of at least about 500. In some aspects, centrifugation is carried out with a relative centrifugal force (G force) of at least about 800. In some aspects, centrifugation is carried out with a relative centrifugal force (G force) of at least about 2000, and in some aspects centrifugation is carried out with a relative centrifugal force of at least about 3000.

In some aspects, centrifugation is carried out for at least about 10 minutes. In some aspects, centrifugation is carried out for at least about 20 minutes, or at least about 30 minutes.

In further aspects, the avocado emulsion or aqueous phase of the avocado emulsion (if heated after centrifugation) is heated at a temperature from about ambient temperature to about 100° C. In some aspects, the avocado emulsion or aqueous phase of the avocado emulsion is heated to at least about 50° C. In various aspects, the avocado emulsion or aqueous phase of the avocado emulsion is heated to about 50° C. to about 85° C. In further aspects, the avocado emulsion or aqueous phase of the avocado emulsion is heated to about 55° C. to about 80° C. In additional aspects, heating of the avocado emulsion or aqueous phase of the avocado emulsion is carried out at a target temperature for at least about 30 seconds. In one aspect, heating is carried out at the target temperature for at least about 1 minute.

In some aspects, heating of the avocado emulsion or the aqueous extract after centrifugation is carried out at a temperature of at least about 40° C. In some aspects, heating is carried out at a temperature of at least about 75° C. In some aspects, heating is carried out at a temperature of at least about 85° C.

In some aspects, heating is carried out for at least about 15 minutes. In some aspects, heating is carried out for at least about 20 minutes. In some aspects, heating is carried out for at least about 30 minutes.

In some aspects, the ratio of water to avocados in the avocado emulsion is at least about 1.5:1 by wet weight. In some aspects, the ratio of water to avocados is at least about 2:1 by wet weight.

In some aspects, the avocados are whole fruit (WF), including the peel and flesh. In some aspects, the avocados are Flesh Only Fruit (FOF), including only the flesh of the avocado, and excluding the peel and seed. In some aspects, the avocados are unripened. In some aspects, the avocados are ripened. In some aspects, the avocados are Hass avocados. In some aspects, the avocados are of a Californian variety. In further aspects, the avocado emulsion is prepared from frozen avocados.

In a particular aspect, the carbohydrate extract or the carbohydrate extract in the process of the disclosure comprises at least about 7% mannoheptulose. In some aspects, the carbohydrate extract comprises at least about 10% mannoheptulose. In some aspects, the carbohydrate extract comprises at least 14% mannoheptulose. In some aspects, the carbohydrate extract comprises at least 18% mannoheptulose. In further aspects, the carbohydrate extract comprises at least 20% mannoheptulose.

In some aspects, the yield of mannoheptulose from the processes described herein is at least about 2 g mannoheptulose per kg avocado or about 0.2% based on the starting mass of the avocados. In particular aspects, the yield of mannoheptulose is at least about 2% based on the starting mass of the avocados. In particular aspects, the yield of mannoheptulose is at least about 4% based on the starting mass of the avocados. In more particular aspects, the yield of mannoheptulose is at least about 8% based on the starting mass of the avocados.

In alternate aspects, the yield of mannoheptulose in the process described herein is at least about 2 g mannoheptulose per kg avocado or about 0.2%. In some aspects, the yield of mannoheptulose is at least about 10 g mannoheptulose per kg avocado or about 1%. In some aspects, the yield of mannoheptulose is at least about 20 g mannoheptulose per kg avocado or about 2%. In some aspects, the yield of mannoheptulose is at least about 40 g mannoheptulose per kg avocado or about 4%. In some aspects, the yield of mannoheptulose is at least about 60 g mannoheptulose per kg avocado or about 6%. In some aspects, the yield of mannoheptulose is at least about 80 g mannoheptulose per kg avocado or about 8%. In some aspects, the yield of mannoheptulose is at least about 100 g mannoheptulose per kg avocado or about 10%.

In one embodiment, the disclosure provides a carbohydrate extract comprising mannoheptulose prepared according to any one of the processes described herein.

In another embodiment, the disclosure provides a process for preparing a food composition comprising combining a carbohydrate extract prepared according to any one of the processes described herein with one or more food composition components. In one aspect, the food composition is a pet food composition.

In one aspect, the disclosure provides a process for preparing a carbohydrate extract comprising mannoheptulose, where the method comprises solubilizing carbohydrates in an avocado emulsion by heating, and separating an aqueous phase from the emulsion by centrifugation to provide a carbohydrate extract comprising at least about 2% mannoheptulose.

For purposes of the invention, the terms “comprising”, “consisting essentially of” and “consisting of” are all given their ordinary meaning, where terms such as including also mean comprising. It is meant that these terms are used interchangeably throughout the application. Thus, as a non-limiting example, where the application states that “the method comprises solubilizing carbohydrates in an avocado emulsion by heating and separating an aqueous phase from the emulsion . . . ” this should be interpreted that the “comprising” language could also interchanged with “consisting essentially of” and/or “consisting of”. Therefore, this passage also supports a claim limitation of “the method consists essentially of solubilizing carbohydrates in an avocado emulsion by heating and separating an aqueous phase from the emulsion . . . ” as well as “the method consists of solubilizing carbohydrates in an avocado emulsion by heating and separating an aqueous phase from the emulsion . . . ” and “the method comprises solubilizing carbohydrates in an avocado emulsion by heating and separating an aqueous phase from the emulsion . . . .”

The preceding summary of the subject matter of the disclosure is supplemented by the following description of various aspects and embodiments of the disclosure, as provided in the following enumerated paragraphs.

Additional aspects, features and variations of the disclosure will be apparent from the entirety of this application, including the detailed description, and all such features are intended as aspects of the disclosure. It should be understood, however, that the detailed description and the specific examples are given by way of illustration, and that the many various changes and modifications that will be apparent to those familiar with the field of the disclosure are also part of the disclosure.

Aspects of the disclosure described with “a” or “an” should be understood to include “one or more” unless the context clearly requires a narrower meaning.

With respect to aspects of the disclosure that have been described as a set or genus, every individual member of the set or genus is intended, individually, as an aspect of the disclosure, even if, for brevity, every individual member has not been specifically mentioned herein. When aspects of the disclosure are described herein as being selected from a genus, it should be understood that the selection can include mixtures of two or more members of the genus. Similarly, with respect to aspects of the disclosure described herein as a range, such as a range of values, every sub-range within the range is considered an aspect of the disclosure.

### DETAILED DESCRIPTION

The disclosure provides an improved process for preparing a carbohydrate extract comprising mannoheptulose and/or perseitol from plants. The process may additionally yield oils and solids useful for other purposes, including, as examples, health and beauty compositions. More specifically, the disclosure provides an improved process for preparing a carbohydrate extract comprising mannoheptulose and/or perseitol from avocados, wherein the process provides extracts with greater yields of mannoheptulose and/or perseitol than previously achieved.

### DEFINITIONS

The term “carbohydrate extract” as used herein is a product prepared by extracting carbohydrates from plant matter and comprises mannoheptulose and/or perseitol along with other plant sugars. In the process of the disclosure, the carbohydrate extract is found in the aqueous phase of the plant emulsion after the emulsion is separated by centrifugation into liquids and solids or into liquids, solids, and oils. The term “carbohydrate extract,” therefore, is used interchangeably in the Examples with the terms “water extract,” or “aqueous phase,” extracted from the plant matter (e.g., avocados) in the process described herein. In some aspects, the carbohydrate extract processed from avocados, as described herein, also comprises polyphenols including, but not limited to, tannins and other anti-oxidants. In some aspects, the carbohydrate extract is measured in terms of degrees Brix. In other aspects, the carbohydrate extract is measured by percent, which is calculated by weight. In aspects when the carbohydrate extract is measured by weight, the extract is dried into a sugar-like solid.

The term “by products” as used herein are products resulting from the centrifugation process of producing the carbohydrate extract where avocado oil and avocado solids are separated from aqueous phase or water extract.

“Brix” is a scale of measurement in the food industry for dissolved solid content in an aqueous solution. For example, degrees Brix (° Bx) refers to, e.g., the sugar content of an aqueous solution. One degree Brix is 1 gram of sugar in 100 grams of solution and represents the strength of the solution as percentage by weight (% w/w). In some aspects of the disclosure, the concentration of the carbohydrate extract is discussed in terms of ° Bx.

All percentages and ratios are calculated by weight unless otherwise indicated. All percentages and ratios are calculated based on the total composition unless otherwise indicated.

As used herein, the term “ambient temperature” is used to indicate a temperature from about 20° C. to about 25° C.

### Processes of the Disclosure

The disclosure is directed to processes for preparing a carbohydrate extract comprising mannoheptulose and/or perseitol from plant matter, food compositions comprising the extract, and to processes for preparing a food composition comprising the extract. In a preferred aspect, the plant matter is avocado. In one aspect, food compositions comprising the carbohydrate extract comprising mannoheptulose and/or perseitol are pet food compositions.

The disclosure provides a process for preparing a carbohydrate extract comprising mannoheptulose and/or perseitol. The process comprises separating an aqueous phase from other phases of an aqueous plant matter emulsion by centrifugation to provide a carbohydrate extract comprising at least about 2% mannoheptulose and/or perseitol. In various embodiments, the plant matter is avocado, alfalfa, fig, primrose, or mixtures thereof. In a preferred aspect, the plant matter is avocado. These plants are known to contain carbohydrate components, such as 6-carbon and 7-carbon sugars. In some aspects, the carbohydrate components include mannoheptulose, 2-deoxy-D-glucose, 5-thio-D-glucose, 3-O-methylglucose, 1,5-anhydro-D-glucitol, or 2,5-anhydro-D-mannitol. See, e.g., U.S. Patent Application Publication Nos. 2002/0035071 and 2005/0249837. If the content of the carbohydrate extract includes a significant amount of perseitol (e.g., at least 10%, or at least 25%, or at least 50%, by weight, of the mannoheptulose and perseitol in the extract), the extract, if desired, may include or may be supplemented with enzymes, such as aldolases, to facilitate the conversion of perseitol to mannoheptulose.

The plant matter, e.g., avocado, may comprise the whole plant or any portion thereof, particularly at least the portion(s) of the plant that contain elevated levels of carbohydrate component.

In various aspects, the plant matter includes the fruit, seed (or pit), branches, leaves, fruit skin, fruit meat, or combination thereof. If the plant matter contains a whole or partial pit, the pit may be optionally removed prior to processing. If the plant matter contains fruit skin, the skin may be optionally removed prior to processing. In various aspects, the emulsion is prepared from whole or partial avocado fruit and water, resulting in an avocado emulsion. In some aspects, the avocado is whole fruit, which includes the pit and peel. In some aspects, the avocado is “flesh only” fruit, which does not include the pit or peel. Alternatively, the emulsion is prepared from avocado flesh and pit, or avocado flesh and skin (without pit). In some aspects, the avocado is frozen. Freezing helps to preserve mannoheptulose and/or perseitol in unripened and ripened avocados. In some aspects, the avocado is cut into pieces or halved prior to freezing. In some aspects, the avocado is fresh. In various aspects, the avocado is ripened, unripened, or the emulsion is prepared using a combination of ripened and unripened avocados. If used, alfalfa, fig, or primrose are similarly processed.

Avocado (also commonly referred to as alligator pear, aguacate, or palta) contains unusually enriched sources of mannoheptulose, as well as related sugars and other carbohydrate components. Avocado is a sub-tropical evergreen tree fruit, growing most successfully in areas of California, Florida, Hawaii, Guatemala, Mexico, Dominican Republic, the West Indies, South Africa, and Asia.

Species of avocado include, for example, Persea Americana and Persea nubigena, including all cultivars within these illustrative species. Cultivars may include ‘Anaheim,’ ‘Bacon,’ ‘Creamhart,’ ‘Duke,’ ‘Fuerte,’ ‘Ganter,’ ‘Gwen,’ ‘Hass,’ ‘Jim,’ ‘Lula,’ ‘Lyon,’ ‘Mexicola,’ ‘Mexicola Grande,’ ‘Murrieta Green,’ ‘Nabal,’ ‘Pinkerton,’ ‘Queen,’ ‘Puebla,’ ‘Reed,’ ‘Rincon,’ ‘Ryan,’ ‘Spinks,’ ‘Topa Topa,’ ‘Whitsell,’ ‘Wurtz,’ and ‘Zutano.’ In various aspects, the aqueous emulsion is prepared from fruit from Persea Americana and/or fruit from cultivars which produce larger fruits (e.g., fruits about 12 ounces or more when the fruit is mature), such as Anaheim, Creamhart, Fuerte, Hass, Lula, Lyon, Murrieta Green, Nabal, Queen, Puebla, Reed, Ryan, and Spinks. In some aspects, tropical avocados are used. “Tropical avocados” are West Indian and West Indian-Guatemalan hybrids which typically have about half the amount of oil compared to Hass Avocados from California or other parts of the world.

Plant matter from alfalfa, fig, or primrose also is reported to provide relatively high levels of mannoheptulose. Alfalfa is also referred to as Medicago sativa. Fig, or Ficus carica (including Cluster fig or Sycamore fig, for example), may also be used in the inventive method, as well as primrose or Primula officinalis.

In one embodiment, production of an emulsified plant mixture comprises combination of the plant matter, e.g., avocado, with an aqueous solution to assist with maceration of the plant into manageable constituents. In one aspect, the aqueous solution is water. In various aspects, the avocado (or other plant matter) and water is mixed at a water to avocado (or other plant matter) ratio, based upon wet weight, of about 1:1, about 1.5:1, about 2:1, about 3:1, about 4:1, about 5:1, about 6:1, about 7:1, about 8:1, about 9:1, about 10:1, about 15:1, and about 20:1. Maceration of the plant breaks down the cells and tissues of the plant into various components. In some aspects, the plant matter is ground or macerated and mixed in water using a food grade emulsifier. In some aspects plant matter is macerated to a particle size of about 100 microns to about 800 microns. In particular aspects, plant matter is macerated to a particle size of about 100 microns, about 200 microns, about 300 microns, about 400 microns, about 500 microns, about 600 microns, about 700 microns, or about 800 microns. In exemplary aspects, plant particle size is about 300 microns. In some aspects, freezing is utilized.

In some aspects, an enzyme having cellulose or pectin activity, or any combination thereof (such as a cellulase, hemicellulase, or pectinase) is included to assist with maceration of the plant. In some aspects, enzymes are used to assist with dissolution and release of carbohydrates from the plant matter (i.e., avocados) via cell wall disruption. Optionally, in some embodiments, the emulsion is pretreated with enzymes that facilitate release of carbohydrates via cell wall disruption. Enzymes are not required in the context of the processes of the disclosure, but may be desirable to accelerate the decomposition of plant matter. In various aspects, the processes described herein are carried out without addition of enzyme.

In some aspects, freezing is utilized. Freezing after maceration stabilizes the ingredients and helps burst cells to increase the release and yield of mannoheptulose.

In some aspects, agitation is utilized. Typically, agitation is carried out for up to about 24 hours, but agitation may be applied to the emulsion process for any length of time suitable to provide an aqueous plant matter emulsion.

In some aspects, the emulsified or digested plant mixture is separated by centrifugation into various phases or fractions. A centrifuge capable of separating liquids (i.e., aqueous phase) from solids is appropriate for use in the context of the disclosure. In some aspects, the centrifuge is a two-phase separator (i.e., a centrifuge that separates an emulsion into liquids and solids). In some aspects, the centrifuge is a three-phase separator (i.e., a centrifuge that separates an emulsion into liquids, solids, and oils). Basket (batch), horizontal or vertical bowl (decanter or tricanter), or vertical disc centrifuges may be used. Basket centrifugation is often used to separate solids from liquids where a screen or filter can be used, most often in a cyclic type operation. Horizontal or vertical bowl centrifugation is often used to separate solids from liquids and, in some aspects, solids from liquids and oils in a continuous type operation. Vertical disc centrifugation is often used to separate solids from liquids and separate different density liquids in a continuous type operation.

In various aspects, separation of the aqueous phase from other phases (e.g., the solid materials and oils) is carried out in a decanter centrifuge or in a tricanter centrifuge. In one aspect, the process comprises separating an aqueous phase from other phases of the avocado emulsion using a decanter, which separates solids from liquids in a slurry or emulsion. The decanter houses a rotating horizontal bowl which has a cylindrical section and a conical section. A scroll is integrated in the bowl. The liquid/solid mixture enters the separating space through a centrally arranged feed tube. The solids are spun against the inner bowl wall under the action of centrifugal force. The scroll, which rotates at a different speed than the bowl shell, transports the solids to the bowl cone. The solids discharge at the end of the bowl through discharge ports. The dry matter content of the solid excrete is variable depending on the total G force and the time subject to this force. Avocado emulsion yields, in various embodiments, are between about 10% and about 35% dry matter content. The liquid is also simultaneously separated. The clarified liquid flows in the opposite direction through the cylindrical section and discharges under gravity.

In one exemplary aspect of the disclosure, a decanter is used to separate the solids from the liquid and then a disk centrifuge is used to separate the final oil and fine solids from the extract. This oil and fine solids can be collected and used in formulation of other products, including, as examples, cosmetics, shampoos, and health products.

In another exemplary aspect of the disclosure, the emulsion is separated into phases via three-phase centrifugal separation, e.g., tricanter separation. In three-phase centrifugal separation, it is possible to separate two liquid phases from one solid phase at the same time. The different densities of the (immiscible) liquids and the solid mean that all three phases can be discharged simultaneously using a tricanter. To ensure separation, the solid phase must be the heaviest phase and the liquid phases must have different densities. Otherwise, the scroll of the decanter will not be able to transport the solid adequately, if at all, thereby affecting the separation result.

The structure and function of a tricanter are similar to those of a decanter (two-phase separation). The decisive difference between these two machines concerns the way that the liquid is discharged. In a tricanter, there are two liquid phases, i.e., a “heavy” liquid phase (higher density and discharged under pressure) as well as a “light” liquid phase (lower density and discharged without pressure). An adjustable impeller discharges the “heavy” liquid phase. An operator of the tricanter can use the adjustable impeller to adjust the pond depth of the heavy liquid without difficulty during ongoing operation. An adjustment mechanism causes the position of the impeller to change, thus changing the separation line of the liquids. The process engineering results can thus be influenced so as to achieve the required separation results.

Any tricanter known in the art can be used to carry out the processes described herein. In exemplary aspects, a TRICANTER® (Flottweg) is used, but the methods of the disclosure are not limited to use of a particular type of centrifuge. Flottweg's TRICANTER® is a horizontal decanter centrifuge for continuous separation of three-phase systems. The TRICANTER® is a countercurrent decanter centrifuge which consists of a cylindrical/conical bowl with a conveyor scroll inside which rotates at a differential speed. The rotating part is driven by electric motors via belt transmission. Feed enters the bowl through a central feed pipe. Through ports in the scroll body, feed passes into the bowl where separation by centrifugal force takes place. In a TRICANTER®, the product is separated into a light liquid phase (such as mineral or olive oil), a heavy liquid phase (such as water), and a solid phase (such as crud, organic residues, and the like). The separated oil is discharged by gravity, while the separated aqueous phase is discharged by an impeller under pressure or by gravity. The separated solids are conveyed by the scroll to the conical end of the bowl and are discharged. The carbohydrate extract (i.e., aqueous phase) comprising mannoheptulose and/or perseitol can be separated and, if desired, further processed or formulated into a food composition.

Centrifugal force used in the processes of the disclosure will vary depending upon the size of the centrifuge and/or centrifuge components and the aqueous emulsion to be separated. In some aspects, the centrifugal force (G) used is at least about 400 G, at least about 500 G, at least about 600 G, at least about 700 G, at least about 800 G, at least about 900 G, at least about 1000 G, at least about 1200 G, at least about 1400 g, at least about 1600 G, at least about 1800 G, at least about 2000 G, at least about 2200 G, at least about 2400 G, at least about 2600 G, at least about 2800 G, at least about 3000 G, at least about 3200 G, at least about 3400 G, at least about 3600 G, at least about 3800 G, at least about 4000 G, at least about 4200 G, at least about 4400 G, at least about 4600 G, at least about 4800 G, at least about 5000 G, at least about 5200 G, at least about 5400 G, at least about 5600 G, at least about 5800 G, at least about 6000 G, at least about 6200 G, at least about 6400 G, at least about 6600 G, at least about 6800 G, at least about 7000 G, at least about 7200 G, at least about 7400 G, at least about 7600 G, at least about 7800 G, at least about 8000 G, at least about 8200 G, at least about 8400 G, at least about 8600 G, at least about 8800 G, at least about 9000 G, at least about 9200 G, at least about 9400 G, at least about 9600 G, at least about 9800 G, at least about 10000 G, at least about 12000 G, at least about 14000 G, at least about 16000 G, at least about 18000 G, and at least about 20000 G. In some aspects, the centrifugal force (G) is between about 500 G and about 10000 G. In some aspects, the centrifugal force is between about 1000 G and about 5000 G. In some aspects, the centrifugal force is between about 2000 G and about 4000 G. In some aspects, the centrifugal force is about 2500 G or about 3500 G. In exemplary aspects, the centrifugal force is about 3000 G.

In some aspects, the centrifugation is carried out for at least about 1 minute, for at least about 2 minutes, for at least about 3 minutes, for at least about 4 minutes, for at least about 5 minutes, for at least about 6 minutes, for at least about 7 minutes, for at least about 8 minutes, for at least about 9 minutes, for at least about 10 minutes, for at least about 11 minutes, for at least about 12 minutes, for at least about 13 minutes, for at least about 14 minutes, for at least about 15 minutes, for at least about 16 minutes, for at least about 17 minutes, for at least about 18 minutes, for at least about 19 minutes, for at least about 20 minutes, for at least about 21 minutes, for at least about 22 minutes, for at least about 23 minutes, for at least about 24 minutes, for at least about 25 minutes, for at least about 26 minutes, for at least about 27 minutes, for at least about 28 minutes, for at least about 29 minutes, for at least about 30 minutes, for at least about 35 minutes, for at least about 40 minutes, for at least about 45 minutes, for at least about 50 minutes, for at least about 55 minutes, for at least about 60 minutes, for at least about 75 minutes, for at least about 90 minutes, for at least about 105 minutes, for at least about 2 hours, for at least about 2.5 hours, for at least about 3 hours, for at least about 4 hours, for at least about 5 hours, for at least about 6 hours, for at least about 7 hours, for at least about 8 hours, for at least about 9 hours, for at least about 10 hours, for at least about 11 hours, for at least about 12 hours, for at least about 13 hours, for at least about 14 hours, for at least about 15 hours, for at least about 16 hours, for at least about 17 hours, for at least about 18 hours, for at least about 19 hours, for at least about 20 hours, for at least about 21 hours, for at least about 22 hours, for at least about 23 hours, and for at least about 24 hours. In particular aspects, centrifugation is carried out from about 1 minute to about 24 hours. In most aspects, centrifugation is carried out from about 1 minute to about 2 hours. In particular aspects, centrifugation is carried out from about 1 minute to about 1 hour. In more particular aspects, centrifugation is carried out from about 1 minute to about 30 minutes. In even more particular aspects, centrifugation is carried out from about 1 minute to about 10 minutes.

In some aspects, liquids, solids, and oils separated by such centrifugation are further separated. Separation techniques include, but are not limited to, gravimetric, centrifugal, filtration, acidification, dehydration, concentration, or combinations thereof. For example, in various aspects, the aqueous phase comprising the carbohydrate extract may be further processed by additional centrifugation, filtration, concentration, drying, or combinations thereof.

In some aspects, the carbohydrate extract comprising mannoheptulose is concentrated, optionally utilizing at least one concentration method selected from the group consisting of heating, vacuum drying, evaporation, refractance window drying, freeze drying, and spray drying, or any combination of the foregoing.

In some aspects, the carbohydrate extract is not dried and is left as a concentrated Brix liquid. In some aspects, Brix liquid has advantages over a dried extract. Those advantages include saving the cost of freeze drying and improving handling of the extract. A dried extract is hygroscopic and picks up moisture easily, which makes it sticky and, in some aspects, makes handling the extract difficult.

In some aspects, a falling film evaporator is used to optimize ° BX of the carbohydrate extract. In particular aspects, the carbohydrate extract, i.e., mannoheptulose syrup, has a ° BX of up to about 50. In various aspects, the ° BX is about 2, is about 3, is about 4, is about 5, is about 6, is about 7, is about 8, is about 9, is about 10, is about 11, is about 12, is about 13, is about 14, is about 15, is about 16, is about 17, is about 18, is about 19, is about 20, is about 21, is about 22, is about 23, is about 24, is about 25, is about 26, is about 27, is about 28, is about 29, is about 30, is about 31, is about 32, is about 33, is about 34, is about 35, is about 36, is about 37, is about 38, is about 39, is about 40, is about 41, is about 42, is about 43, is about 44, is about 45, is about 46, is about 47, is about 48, is about 49, or is about 50. In some aspects, the carbohydrate extract is about 2° BX to about 50° BX. In some aspects, the carbohydrate extract is about 5° BX to about 40° BX. In some aspects, the carbohydrate extract is about 10° BX to about 35° BX. In exemplary aspects, the ° BX of the extract is about 25 to about 35. In more particular aspects, the ° BX of the extract is about 30.

In one embodiment, the inventive process results in enhanced yields of mannoheptulose based on the starting mass of the plant matter (e.g., avocado). Yield of mannoheptulose depends on a number of variables including, but not limited to, the starting material (e.g., whole fruit (pit, peel, and flesh) or flesh-only fruit, and the species or type of avocado), the amount of solids removed, and the amount of water used during processing. Physical losses of mannoheptulose can be as much as about 20% for whole fruit and as much as about 50% for flesh only fruit. Losses can be reduced and yield can be improved by installing a second decanter at the solids exit of the first decanter or tricanter, rewetting the solids and repeating the centrifugation. For example, the yield of mannoheptulose present in the carbohydrate extract subsequent to concentration is as high as about 25%, or from about 0.1% to about 25%, or from about 1% to about 20%, based on the starting mass of the plant matter, e.g., avocados. In various aspects, the yield is about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, or about 25%. In some aspects, the yield is at least about 2 g mannoheptulose per kg avocado or about 0.2%. In some aspects, the yield is at least about 80 g mannoheptulose per kg avocado or about 8%. Of course, even higher yields may be desirable, and lower yields may also be acceptable.

In some aspects, the carbohydrate extract comprising mannoheptulose also comprises natural polyphenols, including tannins, chlorogenic acid, and quinic. Polyphenolic compounds have been shown to have anti-inflammatory, anti-ulcer, and anti-oxidant properties. Tannins are present in plant matter and are highly concentrated in flesh of raw, unripened avocados and in the seed of avocados. In some avocados, the natural polyphenols are present in avocado seed at least at about 13%. By adjusting centrifugal force during the extraction process, levels of these polyphenols in the carbohydrate extract can be increased or decreased.

The process and composition of the disclosure include carbohydrate extracts comprising tannins. In various aspects, tannin content in these extracts is adjusted by controlling the starting material (e.g., unripened avocados and/or unripened avocados comprising pits) or centrifugal force during processing.

In some aspects, heat is used to assist in solubilization of the sugar components from the plant matter into the aqueous phase of the emulsion. In some aspects, water is heated prior to the addition of plant matter, i.e., prior to the preparation of the emulsion. Alternatively or in addition, the water-plant matter mixture is heated during or after maceration or after emulsification. In some aspects, heat is applied after emulsification (e.g., the aqueous emulsion is heated before or during centrifugation). In other aspects, heat is applied after centrifugation to the aqueous phase separated from the solids.

Heat may be increased at the time of, or after, initial heating and agitation to form the solubilized avocado emulsion. In some aspects, the water, emulsion, or aqueous phase is heated in a jacketed tank utilizing low pressure steam to raise temperature. In some aspects, the water, emulsion, or aqueous phase is heated to a temperature of about 20° C. to about 100° C., or from about 30° C. to about 90° C., or from about 40° C. to about 85° C., or from about 50° C. to about 80° C., or from about 55° C. to about 75° C., or from about 60° C. to about 75° C., or from about 65° C. to about 70° C. In some aspects, the water, emulsion, or aqueous phase is heated to a temperature of at least about 40° C., at least about 45° C., at least about 50° C., at least about 55° C., at least about 60° C., at least about 65° C., at least about 70° C., at least about 75° C., at least about 80° C., at least about 85° C., at least about 90° C., at least about 95° C., or at least about 100° C. In some aspects, the heating of the emulsion is continuous. In some aspects, the heating of the emulsion is spiked.

In some aspects, the emulsion, or the aqueous phase separated from the emulsion, is heated. A heating step is often used to solubilize sugars and, in some instances, to kill microbes. In various aspects, heating is carried out for at least about 1 minute, for at least about 1.5 minutes, for at least about 2 minutes, for at least about 2.5 minutes, for at least about 3 minutes, for at least about 3.5 minutes, for at least about 4 minutes, for at least about 4.5 minutes, for at least about 5 minutes, for at least about 6 minutes, for at least about 7 minutes, for at least about 8 minutes, for at least about 9 minutes, for at least about 10 minutes, for at least about 11 minutes, for at least about 12 minutes, for at least about 13 minutes, for at least about 14 minutes, for at least about 15 minutes, for at least about 16 minutes, for at least about 17 minutes, for at least about 18 minutes, for at least about 19 minutes, for at least about 20 minutes, for at least about 21 minutes, for at least about 22 minutes, for at least about 23 minutes, for at least about 24 minutes, for at least about 25 minutes, for at least about 26 minutes, for at least about 27 minutes, for at least about 28 minutes, for at least about 29 minutes, for at least about 30 minutes, for at least about 35 minutes, for at least about 40 minutes, for at least about 45 minutes, for at least about 50 minutes, for at least about 55 minutes, for at least about 60 minutes, for at least about 75 minutes, for at least about 90 minutes, for at least about 105 minutes, for at least about 2 hours, for at least about 2.5 hours, for at least about 3 hours, for at least about 4 hours, for at least about 5 hours, for at least about 6 hours, for at least about 7 hours, for at least about 8 hours, for at least about 9 hours, for at least about 10 hours, for at least about 11 hours, for at least about 12 hours, for at least about 13 hours, for at least about 14 hours, for at least about 15 hours, for at least about 16 hours, for at least about 17 hours, for at least about 18 hours, for at least about 19 hours, for at least about 20 hours, for at least about 21 hours, for at least about 22 hours, for at least about 23 hours, and for at least about 24 hours. In some aspects, heating is carried out for about 1 minute to about 24 hours. In some aspects, heating is carried out for about 1 minute to about 4 hours. In some aspects, heating is carried out for about 1 minute to about 1 hour. In another aspect, heating is carried out for about 1 minute to about 30 minutes. In a further aspect, heating is carried out for about 1 minute to about 10 minutes.

In some aspects, heat is added to avocado emulsion or to the aqueous extract of the avocado emulsion to inactivate plant/fruit enzymes that cause ripening, rancidity or deterioration of fruit quality. In some aspects, the composition is sonicated to inactivate enzymes. In some aspects, the composition is treated with supercritical carbon dioxide (SC—CO₂) to inactivate enzymes and kill microbes.

In some aspects, the pH is controlled to preserve enzyme activity, often in the range of pH from about 3 to about 7, in the range from about 3.5 to about 6.5, in the range from about 4 to about 6, and sometimes in the range from about 4.5 to about 5.5. In other aspects the pH is about 2.0, about 2.1, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.6, about 5.7, about 5.8, about 5.9, about 6.0, about 6.1, about 6.2, about 6.3, about 6.4, about 6.5, about 6.6, about 6.7, about 6.8, about 6.9, and about 7.0.

In some aspects, the pH is controlled to prevent bacterial growth, often in the range of pH from about 2.5 to about 6, in the range from about 3.0 to about 5.5, and sometimes in the range from about 3.5 to about 5.0. In particular aspects, the process described herein comprises lowering pH to at most about pH 6.0, about pH 5.9, about pH 5.8, about pH 5.7, about pH 5.6, about pH 5.5, about pH 5.4, about pH 5.3, about pH 5.2, about pH 5.1, about pH 5.0, about pH 4.9, about pH 4.8, about pH 4.7, about pH 4.6, about pH 4.5, about pH 4.4, about pH 4.3, about pH 4.2, about pH 4.1, about pH 4.0, about pH 3.9, about pH 3.8, about pH 3.7, about pH 3.6, about pH 3.5, about pH 3.4, about pH 3.3, about pH 3.2, about pH 3.1, about pH 3.0, about pH 2.9, about pH 2.8, about pH 2.7, about pH 2.6, about pH 2.5, about pH 2.4, about pH 2.3, about pH 2.2, about pH 2.1, or about pH 2.0. In exemplary aspects, the process described herein comprises lowering pH to about pH 3.8 to, e.g., prevent Salmonella growth.

In some aspects, the inventive process comprises an ultrafiltration step, although ultrafiltration is not required. Ultrafiltration is a membrane separation process driven by a pressure gradient. The membrane separates liquid components according to their size and structure. Ultrafiltration removes larger molecules, like polyphenoloxidases, but does not remove lower-molecular-weight components like polyphenols.

In some aspects, the process further comprises a nanofiltration step. Nanofiltration is a membrane filtration process used to separate a range of inorganic and organic substances from solution. This filtration process is carried out by diffusion through a membrane, under pressure differentials that are considerably less than pressure differentials used in reverse osmosis, but still significantly greater than those in ultrafiltration. Nanofiltration has a unique ability to separate and fractionate ionic and relatively low molecular weight organic species. In some aspects, however, the process of the disclosure is carried out without nanofiltration.

In some aspects, the inventive process comprises a pasteurization step. Pasteurization is a process of heating a liquid (or food) to a specific temperature for a predefined length of time and then immediately cooling it. Pasteurization slows spoilage caused by microbial growth in the food.

In some aspects, the process of the disclosures includes a step of concentrating and/or drying the extract. All methods of concentrating and/or drying carbohydrate extracts are contemplated for use in the processes described herein. In some aspects, the drying is used to concentrate the carbohydrate extract. In various aspects, the process of concentrating or drying the extract is carried out by heating, vacuum drying, evaporating, refractance window drying, freeze drying, or spray drying. In one aspect, the drying is freeze-drying. Freeze-drying, also known as lyophilization, or cryodesiccation, is a dehydration process typically used to preserve a perishable material or make the material more convenient for transport. Freeze-drying works by freezing the material and then reducing the surrounding pressure to allow the frozen water in the material to sublimate directly from the solid phase to the gas phase. The carbohydrate extract described herein is optionally freeze-dried.

In various aspects, the process described herein comprises the following steps: mixing avocados with water, grinding the avocados and water into an emulsion, centrifuging, heating the aqueous phase, filtering using ultrafiltration, filtering using nanofiltration, and concentrating and/or drying the filtrate (by, e.g., heating, vacuum drying, evaporating, refractance window drying, freeze drying, or spray drying) to obtain a carbohydrate extract comprising mannoheptulose. In various aspects, some of these steps are unnecessary and carbohydrate extracts are made without them. For example, in some embodiments, one or more of the steps of heating, ultrafiltration, nanofiltration, freeze-drying, and/or other means of drying are eliminated. In further aspects, the order of the steps is changed. For example, when preparing extracts from avocados, and particularly whole fruit, it is sometimes preferable to centrifuge the emulsion before heating. In some aspects, however, it may be preferable to heat the emulsion before centrifugation. In additional aspects, it is sometimes preferable to carry out two or more steps of centrifugation to improve the quality of the extract and obtain increased levels of mannoheptulose.

In various aspects, the process described herein provides a carbohydrate extract that comprises at least about 1% mannoheptulose, at least about 2% mannoheptulose, at least about 3% mannoheptulose, at least about 4% mannoheptulose, at least about 5% mannoheptulose, at least about 6% mannoheptulose, at least about 7% mannoheptulose, at least about 8% mannoheptulose, at least about 9% mannoheptulose, at least about 10% mannoheptulose, at least about 11% mannoheptulose, at least about 12% mannoheptulose, at least about 13% mannoheptulose, at least about 14% mannoheptulose, at least about 15% mannoheptulose, at least about 16% mannoheptulose, at least about 17% mannoheptulose, at least about 18% mannoheptulose, at least about 19% mannoheptulose, at least about 20% mannoheptulose, at least about 21% mannoheptulose, at least about 22% mannoheptulose, at least about 23% mannoheptulose, at least about 24% mannoheptulose, at least about 25% mannoheptulose, at least about 26% mannoheptulose, at least about 27% mannoheptulose, at least about 28% mannoheptulose, at least about 29% mannoheptulose, at least about 30% mannoheptulose, at least about 35% mannoheptulose, at least about 40% mannoheptulose, at least about 45% mannoheptulose, and at least about 50% mannoheptulose. In some aspects, the carbohydrate extract comprises between about 1% mannoheptulose and about 40% mannoheptulose. In further aspects, the carbohydrate extract comprises between about 2% mannoheptulose and about 30% mannoheptulose. In particular aspects, the carbohydrate extract comprises between about 2% mannoheptulose and about 25% mannoheptulose. In various aspects, yields from whole, unripened avocados will be much higher than yields from ripened, flesh-only avocados. In some aspects, yields from tropical avocados will be higher than yields from avocados that have a much higher fat or oil concentration, i.e., Hass avocados.

In various aspects, the amount of mannoheptulose obtained by the process described herein depends upon the amount of mannoheptulose in the avocado starting materials. For example, unripened avocados contain greater amounts of mannoheptulose than ripened avocados, and certain species, cultivars (or varieties) of avocados comprise greater amounts of mannoheptulose than others.

In various aspects, the process described herein is analyzed based upon mass of mannoheptulose after drying per starting mass of avocados. In other words, yield of mannoheptulose is based upon mass of mannoheptulose per starting mass of avocados. In various aspects, the yield is about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100, about 1:110, about 1:120, about 1:130, about 1:140, about 1:150, about 1:160, about 1:170, about 1:180, about 1:190, about 1:200, about 1:210, about 1:220, about 1:230, about 1:240, about 1:250, about 1:260, about 1:270, about 1:280, about 1:280, about 1:290, about 1:300, about 1:310, about 1:320, about 1:330, about 1:340, about 1:350, about 1:360, about 1:370, about 1:380, about 1:400, about 1:450, or about 1:500. In various aspects, the yield ranges from about 1:50 to about 1:400, or about 1:100 to about 1:350, or about 1:150 to about 1:250.

### Compositions of the Disclosure

The disclosure provides compositions comprising the carbohydrate extract comprising mannoheptulose and/or perseitol, such as the carbohydrate extract prepared as described herein. Compositions comprising the carbohydrate extract are useful, for example, to alter glucose (or other energy source) utilization and/or mimic metabolic effects of caloric restriction. Caloric restriction has been consistently shown to extend longevity in animals. See Weindruch and Walford, “The Retardation of Aging and Disease by Dietary Restriction,” Springfield, Ill.: Charles C. Thomas (1988); Yu, “Modulation of Aging Processes by Dietary Restriction,” Boca Raton: CRC Press (1994); and Fishbein, “Biological Effects of Dietary Restriction,” Springer, New York (1991).

The disclosure includes a food composition, i.e., a composition that is intended for ingestion by an animal, such as a human, or other animal (including a pet), comprising the carbohydrate extract. Pet food compositions may include, without limitation, nutritionally balanced compositions suitable for daily feed, as well as supplements (e.g., treats) which may or may not be nutritionally balanced. As used herein, the term “nutritionally balanced,” with reference to the pet food composition, means that the composition has known required nutrients to sustain life in proper amounts and proportion based on recommendations of recognized authorities in the field of pet nutrition.

In one embodiment herein, the process of the disclosure is utilized to prepare a food composition comprising a carbohydrate extract comprising mannoheptulose from avocados. In some aspects, the carbohydrate extract comprises mannoheptulose and other sugars of avocado. In some aspects, the carbohydrate extract comprises a component selected from mannoheptulose, 2-deoxy-D-glucose, 5-thio-D-glucose, 3-O-methylglucose, 1,5-anhydro-D-glucitol, 2,5-anhydro-D-mannitol, and mixtures thereof.

The level of carbohydrate extract present in the composition can be determined based on desired physiological or nutritional response to the extract. Relatively low doses and relatively high doses of the carbohydrate extract may be useful in some embodiments, while providing less than optimal efficacy for others. A desired dose to a pet, on a daily basis, has been discovered to be from about 1 mg/kg to about 15 mg/kg, such as from about 2 mg/kg to about 10 mg/kg, e.g., from about 2 mg/kg to about 5 mg/kg, wherein (as will be commonly understood in the art) the “mg” refers to level (weight) of the component and the “kg” refers to kilograms (weight) of the pet. Such desired dose is optimal in some pet diets as a calorie restriction mimetic that delivers anti-aging and health-promoting benefits of calorie restriction without reducing food intake. In certain embodiments, this may translate to preparation of pet food compositions comprising less than about 5%, or less than about 2%, or from about 0.0001% to about 0.5% of the carbohydrate extract, all by weight of the composition. The level of carbohydrate extract in the composition may be determined by one of ordinary skill in the art based on a variety of factors, for example, the form of the pet food composition (e.g., whether a dry composition, semi-moist composition, wet composition, or supplement, or any other form or mixture thereof). The ordinarily skilled artisan will be able to utilize the doses provided herein, and use these to determine the optimal level of carbohydrate extract within a given pet food composition.

Food compositions will contain one or more additional components, such as components that supply necessary dietary requirements, as well as treats (e.g., dog biscuits) or other food supplements. Optionally, the composition herein is a pet food composition, such as a dry composition (for example, kibble), semi-moist composition, wet composition, or any mixture thereof. Alternatively or additionally, the composition is a supplement, such as a gravy, drinking water, yogurt, powder, suspension, chew, treat (e.g., biscuits) or any other delivery form.

In one embodiment, the food composition may comprise, on a dry matter basis, from about 10% to about 90% crude protein, alternatively from about 20% to about 50% crude protein, alternatively from about 20% to about 40% crude protein, by weight of the food composition, or alternatively from about 20% to about 35% crude protein. The crude protein material may comprise vegetable proteins such as soybean, cottonseed, and peanut, or animal proteins such as casein, albumin, and meat protein. Non-limiting examples of meat protein useful herein include a protein source selected from the group consisting of beef, pork, lamb, poultry, fish, vegetable, and mixtures thereof.

Furthermore, the compositions may comprise, on a dry matter basis, from about 5% to about 40% fat, alternatively from about 10% to about 35% fat, by weight of the food composition.

The compositions of the present disclosure may further comprise an additional carbohydrate source. Grains or cereals such as rice, corn, milo, sorghum, barley, wheat, and the like are illustrative sources.

The compositions may also contain one or more other materials such as dried whey and other dairy by products.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Additional aspects and details of the invention will be apparent from the following examples, which are intended to be illustrative rather than limiting.

### Example 1

### Determining Manufacturing Processes for Obtaining Increased Concentrations of Mannoheptulose from Avocados

Experiments were carried out to determine new and improved processes for making carbohydrate extracts comprising increased concentrations of mannoheptulose from flesh-only avocados and whole fruit avocados.

Three lots of avocados were utilized during this testing: 1) Mexican avocados, unripened whole fruit from middle season; 2) Mexican avocados, ripened, flesh-only fruit with no peel or pit (halves) packed in plastic bags; and 3) Chilean avocados, unripened whole fruit from middle season from the Hass variety. The mannoheptulose levels of each of the three lots of avocados are listed in Table 1, which shows that unripened avocados comprised greater amounts of mannoheptulose.

TABLE 1
Avocado Raw Materials with Mannoheptulose (MH) in ppm

| Avocados | Description | MH ppm |
|---|---|---|
| Mexican Hass | Whole fruit, diced and | 6,342 |
| | frozen | |
| Mexican Hass | Flesh only fruit (no peel | 2,020 |
| | or pit), ripened, cut in | |
| | halves and frozen | |
| Chilean Hass | Whole fruit, diced and | 6,073 |
| | frozen | |

Whole avocados were pre-processed and frozen until testing. Pre-processing included the steps of removal of stickers by hand and chopping fruit in small pieces. The halves were frozen by the supplier and kept frozen until tests were conducted.

An objective of the study was to identify process features that increase recovery of mannoheptulose.

Some features of the production process are set out below:

1. The pH of the extract was controlled at pH 3.8 with addition of citric acid during the step of heating/extraction to prevent microbial growth (including preventing growth of Salmonella).

2. The heating/extraction step was modified to reduce the residence time from 1 hr to 45 min and the temperature was reduced from 85° C. to 75° C. The control of pH was added as a preventive step for microbial growth, because the reduced temperature can still accomplish the killing of microbes and the deactivation of enzymes (e.g., polyphenol oxidases (PPO), pectin esterases (PE), cellulases, and lipases).

3. The centrifugation step was carried out prior to the heating step. Carrying out centrifugation prior to heating allowed for the effective separation of pulp (mostly insoluble fiber) and oil from the water soluble components (including mannoheptulose). Separation of pulp/flesh prior to heating avoided gelatinization of small levels of starch present in pulp that may increase viscosity of the slurry and bind the oil and/or mannoheptulose, thereby decreasing yield. Additionally, separation of pulp prior to heating increased effectiveness of the heating step; time to achieve target temperature and cooling time was reduced significantly once the flesh was removed from the water soluble extract.

4. No further processing was performed using separation steps other than centrifugation, such as microfiltration and nanofiltration.

5. Different approaches for making the carbohydrate extract comprising mannoheptulose were employed. In some aspects, steps in the processes included (1) mixing the avocados with water, (2) grinding the avocados and water into an emulsion, (3) centrifugation, (4) heating, (5) ultrafiltration, (6) nanofiltration, (7) freeze-drying, and/or (8) spray-drying, vacuum drying, and the like. In various aspects, some of these steps are unnecessary and carbohydrate extracts can be made without them, but possibly at decreased yield, especially if the heating step is removed. For example, in some processes, one or more of the steps of heating, ultrafiltration, nanofiltration, freeze-drying, and/or other means of drying are eliminated. In some aspects, the order of the steps is changed to increase yield of mannoheptulose in the carbohydrate extract.

### Data

During the production of the extracts (PP1 and PP2), samples were collected to estimate mannoheptulose levels after processing. Preliminary data was collected to calculate a mass balance during different runs. Samples collected during the runs were representative of different approaches used to make the extracts (e.g., flesh only fruit and/or whole fruit). The process denoted “Process Extract—control” comprised heating the emulsion, centrifugation, and ultrafiltration of the resulting isolated aqueous phase. The process denoted “Process Extract #1” comprised centrifuging the emulsion, heating the isolated aqueous phase, and purifying the aqueous phase via ultrafiltration. The process denoted “Process Extract #2” comprised centrifuging the emulsion without a heating or ultrafiltration step. The process denoted “Process Extract #3” comprised centrifuging the emulsion and heating the isolated aqueous phase, but did not entail an ultrafiltration step. The following data was collected during the test: Brix, color, and dry matter. Additional data collected included % mannoheptulose (MH), % fat content, and % moisture content (MC). See Table 2.

TABLE 2
Yield of Mannoheptulose (MH) in Various Extracts

| | | | | P&G | | | |
|---|---|---|---|---|---|---|---|
| | | | | (% dry weight | | | |
| | | | % MC | MH in the | | | |
| Avocado | % MH | Batch | (Moisture | carbohydrate | % Fat | | |
| Type | Avocados | # | Content) | extract) | (wt/wt) | Yield | Description |
| Calavo | 0.61 | 12/9 M | 15.51 | 14.4 | 2.3 | 121:1 | Process Extract - |
| whole, | | | | | | | control: Heating, |
| unripened | | | | | | | Centrifugation, and |
| | | | | | | | Ultrafiltration, no |
| | | | | | | | microfiltration or |
| | | | | | | | nanofiltration. |
| Mexico | 0.22 | 12/10 M | 16.8 | 7.7 | 2.7 | 186:1 | Process Extract - |
| Ripened, | | | | | | | control: Heating, |
| flesh | | | | | | | Centrifugation, |
| only | | | | | | | Ultrafiltration, no |
| | | | | | | | microfiltration or |
| | | | | | | | nanofiltration. |
| Mexico | 0.22 | 12/14 M | 14.94 | 5.3 | 2.04 | 282:1 | Process Extract #1 |
| Ripened, | | | | | | | (centrifugation, |
| flesh | | | | | | | heating, |
| only | | | | | | | ultrafiltration) |
| Mexico | 0.22 | 12/14 D 7M | 12.23 | 2.4 | 24.9 | NA | Process Extract #2 |
| Ripened, | | | | | | | (centrifugation, no |
| flesh | | | | | | | heating, no |
| only | | | | | | | ultrafiltration) |
| Mexico | 0.22 | 12/14 EM | 11.95 | 2.4 | 27.6 | NA | Process Extract #3 |
| Ripened, | | | | | | | (centrifugation, |
| flesh | | | | | | | heating, no |
| only | | | | | | | ultrafiltration) |
| Calavo | 0.61 | 12/15 M | 17.0 | 17.8 | 1.9 | 162:1 | Process Extract # 1 |
| whole, | | | | | | | (centrifugation, |
| unripened | | | | | | | heating, |
| | | | | | | | ultrafiltration) |
| Calavo | 0.61 | 12/15 D6 M | 14.36 | 10.7 | 21.3 | NA | Process Extract #2 |
| whole, | | | | | | | (centrifugation, no |
| unripened | | | | | | | heating, no |
| | | | | | | | ultrafiltration) |
| Calavo | 0.61 | 12/15 EM | 14.16 | 11.0 | 21.0 | NA | Process Extract #3 |
| whole, | | | | | | | (centrifugation, |
| unripened | | | | | | | heating, no |
| | | | | | | | ultrafiltration) |

### Results

A carbohydrate extract comprising >13% mannoheptulose was produced using whole fruit avocado starting material comprising 0.61% mannoheptulose per wet weight of avocado. Flesh only (FO) avocados comprising about 0.22% mannoheptulose wet weight yielded extracts with mannoheptulose levels as high as 7.7%. These results demonstrate that the extraction method and starting material affects yield. The yield using whole fruit was 121:1 kg of avocados/kg of extract.

The carbohydrate extract comprising the greatest amount of mannoheptulose (˜18%) and lowest fat content was made with whole, unripened avocados, using a process comprising centrifugation, heating, and ultrafiltration steps, in order (see Table 2). This high level of mannoheptulose was achieved by separating most of the oil and flesh during centrifugation prior to the heating step. Without being bound by any particular theory, it is thought that, although heating frees the oil from the emulsion more effectively, heating also increases the viscosity of the slurry, which complicates separation of water-soluble compounds during the centrifugation step. Additionally, the time to achieve target temperature required to reduce microbial load and/or inactivate enzymes is reduced when the heating step is performed after centrifugation, on clear liquid lacking fiber and oil. Heating after centrifugation also may minimize caramelization (sugar-sugar reaction) and Maillard reactions (amino acid and sugar reaction), which may contribute to mannoheptulose losses. Thus, in some embodiments, the inventive process comprises the steps of (a) centrifugation, then (b) heating, and (optionally) (c) ultrafiltration, which showed advantages over the control process (heating, centrifugation, and ultrafiltration, as described above), particularly when whole fruit was used as starting material.

When FO avocados were used to create an aqueous emulsion, centrifugation prior to heating did not increase the level of mannoheptulose in the finished extract. See “Process Extract #3” in Table 2; the process did not include ultrafiltration.

The results from Process Extract #2 (no heating, no ultrafiltration) and Process Extract #3 (no ultrafiltration) demonstrated that a carbohydrate extract comprising mannoheptulose can be made by only separating the aqueous phase from other phases in the emulsion by centrifugation. However, the yield was reduced compared to Process Extract #1 (centrifugation, then heating, then ultrafiltration).

Various features of the production process described above may be varied to achieve a desired yield, as set out in Table 3 below.

TABLE 3
Features of the extraction process

| Process Step | Transformation | Observations |
|---|---|---|
| Grinding/ | Promotes separation of | May be advantageous to use |
| Mixing | oil - breaking cells to | chopped, frozen avocados vs. |
| | liberate oil and water | whole, frozen avocados as |
| | from the avocados. | starting material in some |
| | | embodiments. |
| Heating/ | 85° C. for 1 hr, | May be advantageous to |
| Control pH | pH 3.8 to control | reduce time and temperature |
| | Salmonella | (75° C. for 30 min) to reduce |
| | | sugar reactions. |
| Centrifugation | Separation of water from | In some processes, |
| | fruit flesh and oil. | centrifugation performed |
| | | before heating. |
| Microfiltration | De-oiling | Optional; centrifugation |
| | | separates most of the oil from |
| | | the flesh and water. |
| Ultrafiltration | 10 KDa | Optional; may be desirable to |
| | Further separation of | remove proteins and other |
| | soluble compounds with | small MW compounds, assists |
| | low molecular weight, | fat removal, particularly for |
| | such as proteins | FO extract. |
| Nanofiltration | Dewatering | Optional |
| Microfiltration/ | 100 KDa separation of | Optional; level of suspended |
| Polishing | particles | solids is controlled during |
| | | early stages of the process. |
| Freeze-Drying | Water removal | |

### Observations

Differences between whole fruit and flesh only extracts were observed from the initial steps of the process. The whole fruit slurry browned faster and developed a dark brown color, while the flesh only extract was lighter in color. The pits comprise tannins and are the source of pigments coloring the extract. After pH adjustment, browning was decreased resulting in a light green emulsion, while flesh only slurry was a light beige.

Recovery data demonstrated that the maximum level of mannoheptulose in recovered carbohydrate extract was achieved after centrifugation and heating (or heating and centrifugation), followed by ultrafiltration. Centrifugation removes the insoluble material and most of the oil, specifically in the case of the whole fruit extract. When heating was carried out before centrifugation, using a whole fruit avocado emulsion, percent recovery of mannoheptulose dropped significantly. Without being bound by any particular theory, one potential reason for this lower recovery is increased emulsion viscosity due to gelatinization of pectin and starch during heating. After heating, the emulsion (emulsified mixture of avocado, water and oil) is thick due to the presence of avocado oil. In the case of flesh only extracts, performing a centrifugation step before a heating step did not have the same effect as that observed for the whole fruit extract. In fact, no change in mannoheptulose level was observed. Without wishing to be bound by theory, this may be due to the different composition of the FOF and WF extracts, due to the absence of pit and peel, which contribute pectins and/or other polysaccharides. When working with WF, there is a benefit to performing a centrifugation step before heating. When working with FOF, performing a centrifugation step before heating did not offer additional benefit.

The rate limiting step for the extraction processes is the freeze-drying step, because freeze-drying can take five days or more to carry out depending on the sugar content of the material. Alternative methods for drying carbohydrate extracts are suitable for inclusion in the process described herein.

### Example 2

### Determining New Processes for Obtaining Increased Concentrations of Mannoheptulose from Flesh Only and Whole Fruit Avocados

Experiments were carried out to determine process features to obtain (1) at least 5 kg of carbohydrate extract comprising mannoheptulose from flesh only avocados at a concentration greater than 5%, and (2) at least 25 kg of carbohydrate extract comprising mannoheptulose from whole fruit avocados at a minimum concentration of 20%.

In this experiment, Californian whole fruit avocados (unripened) and Mexican frozen avocado halves (ripened, no peel or pit) were obtained from Calavo Growers (Houston, Tex.). Both were Hass avocados. The pre-processing of the avocados included removing stickers by hand and chopping the avocados in small pieces.

TABLE 4
Avocado Raw Materials with Mannoheptulose Results in ppm

| Avocados | Description | MH ppm |
|---|---|---|
| Californian Hass | Whole fruit, unripened | 16,220 |
| Mexican Hass | Cut in halves and frozen, ripened | 2,020 |
| | (no peel or pit) | |

Whole Californian Hass avocados were received in boxes and immediately cut in wedges with ˜½ in thickness. The avocado wedges were immediately frozen and kept in the freezer until the testing. Whole sliced avocados were allowed to partially melt overnight. Partially frozen avocados were blended with water (2:1 ratio water:avocados) and ground to a slurry with a very fine particle size distribution (PSD).

Centrifugation was carried out prior to heating. Centrifuging prior to heating ensured effective separation of the pulp (mostly insoluble fiber) and oil from the water-soluble components (including mannoheptulose) prior to heating. The objective was to avoid any gelatinization of small levels of starch present in the pulp that might increase viscosity of the slurry and bind the oil and/or mannoheptulose and, therefore, decrease mannoheptulose yield. Additionally, heating of the water-soluble components is carried out more effectively when the pulp is removed because the time to achieve the target temperature and cooling is reduced without the pulp.

The pH of the extract was controlled at pH of 3.8 with addition of citric acid during heating/extraction to prevent microbial growth (including Salmonella).

The heating step was modified to reduce the residence time from 1 hr to 45 min and the temperature from 85° C. to 75° C. Since pH was adjusted to as a preventive step for microbial growth, the process could be performed at a reduced temperature while still accomplishing the kill step and deactivation of enzymes (e.g., PPO, PE, cellulases, and lipases).

A nanofiltration step was included in the process to remove part of the water from the carbohydrate extract prior to drying. At the exit of the nanofiltration, the concentration of the liquid carbohydrate extract was 25° Brix.

### CONCLUSION

Whole fruit extract was produced with mannoheptulose levels of about 25% mannoheptulose. Flesh only extract was produced with a concentration of 10% mannoheptulose.

### Example 3

### Determining Tricanter Equipment G Force Needed to Separate Avocado Sugars, Oil, and Solids

Experiments were carried out to determine the centrifugal force needed to separate an emulsion comprising avocados and water into different phases, e.g., phases comprising fats (oil phase), solids, floating solids, and sugars (aqueous phase).

Three different raw material preparations were made as follows:

(1) Ground, unripe, frozen, whole avocados mixed 1 to 1 by weight with water;
(2) Ground, unripe, “next season”, frozen, whole avocados mixed 1 to 1 by weight with water (“Next season” avocados are not yet mature; an avocado tree can carry two seasons of fruit: the first season (ready-to-pick) and the next season (developing fruit which are not yet mature)); and
(3) Finely ground, ripe, ready-to-eat, whole avocados mixed 1 to 1 by weight with water.

### Unripe, Frozen, Whole Avocados

The first product tested was the ground, frozen, whole avocado. Total sample was heated to about 160 degrees Fahrenheit. Fifteen ml of the product sample was put in a conical centrifuge test tube and was subjected to a G force of 2000 for 1 minute. The sample was separated as follows:

0.6% oil

### 18.6% Solids

18.6% floating solids
62.2% Water extract (turbid)

No sharp separation was seen. Water was turbid with floating solids and some oil was visible in the floating solids. The same sample was subjected again to another minute of centrifugation using 2000 G, which had no effect on the aqueous phase. A third centrifugation did not change the data set out above.

A new sample (test tube) was prepared and was subjected to one minute of centrifugation at 3000 G. The sample was separated as follows:

1% oil

### 13% Solids

32% floating solids
54% Water extract

By increasing centrifugal force, the percentage of floating solids in the carbohydrate extract (the aqueous phase) was much higher. This increase in floating solids was most likely due to non-homogeneity in the sample as collected. When subjected again to the same centrifugation conditions, the aqueous phase became much clearer. This test showed that, when using a tricanter in the production process under these conditions, a tricanter capable of applying at least 3000 G is preferable.

### Unripe, “Next Season” Frozen, Whole Avocados

The second product tested was the ground, unripe, “next season” frozen, whole avocados. The total sample was heated to around 160 degrees Fahrenheit and 15 ml of product sample was put into a conical centrifuge test tube. The sample was subjected to a G force of 2000 for 1 minute. The sample was separated as follows:

0% oil (0% oil was expected since unripe or non-mature fruit was used. Avocado fruit obtains oil after maturity and ripening.)
20% solids
20% floating solids (also called “rag” in the industry)
60% water extract (turbid with floating solids)

The same sample was then subjected again to another minute of centrifugation at 2000 G, which had no effect. A third subjection of the sample to centrifugation did not change the above data.

Next, the ground unripe, “next season” frozen, whole avocado was tested in this process. However, in this experiment, centrifugation was carried out with a G force of 3000. Results were similar to the test with a G force of 2000. A second subjection to centrifugation at 3000 G did not result in substantial improvement. Floating solids were still seen in the water extract (or aqueous phase), which exhibited a slight oil film. Apparently some oil was already present in these unripened avocados. Because of the presence of the floating solids in the aqueous phase, the sample was subjected to centrifugation at 10,000 G. However, the increased force did not provide better results.

From this point forward, samples comprising ground, unripe, “next season” frozen, whole avocados were diluted with an extra 50% water addition by weight. The additional water resulted in a 2:1 (2 parts water:1 part avocado) mixture. The sample was heated to about 160 degrees Fahrenheit. Fifteen ml of product sample was put in a conical centrifuge test tube and was subjected to a G force of 3000 for 1 minute. The diluted sample had an increased aqueous phase, which was much clearer than the less dilute sample, without floating solids or oil in the water extract.

### Ripened, Whole Avocados

The third product tested was finely ground, ripe, ready-to-eat, whole avocados. The sample was prepared and heated to 82 degrees Fahrenheit (27.78° C.) to determine if this low temperature would allow good separation of the layers/phases. Fifteen ml of product sample was put into a conical centrifuge test tube and was subjected to a G force of 3000 for 1 minute. The results were as follows:

0% oil
35% floating solids
17% water extract
48% solids
No oil extraction occurred due to the low temperature.

Next, the finely ground, ripe, ready-to-eat, whole avocado was prepared and heated to 160 degrees Fahrenheit. Fifteen ml of product sample was put into a conical centrifuge test tube and was subjected to a G force of 3000 for 1 minute. The results were as follows:

3.2% oil
35% floating solids
25% water extract
35% solids
Interestingly, the floating solids stayed the same but the bottom solids were reduced and the water extract yield increased.

Finely ground, ripe, ready-to-eat, whole avocados were then prepared at a ratio of 2:1 (water to avocado) and heated to 170 degrees Fahrenheit (76.67° C.). Fifteen ml of sample was placed into a conical centrifuge test tube and was subjected to a G force of 3000 for 1 minute. The results were as follows:

1.5% oil
20% floating solids
53% water extract
27% solids

The water extract was doubled compared to the previous sample comprising a 1:1 ratio of water to avocado. The percentage of oil was about halved. The additional water in the mixture allowed better separation of the fibers from the aqueous phase. Thus, floating solids grew and could be due to hydrolysis. To see if that was the case an acidification of the product sample was performed.

The finely ground, ripened, whole avocado sample was diluted 1 to 1 again with water. The sample also was acidified with muriatic acid, starting from a pH of 6.5 and reduced to a pH of 3.4. Acidification supports microbial shelf stability and allows better separation of solids and liquids. The sample also was heated to 158 degrees Fahrenheit (70° C.). A 15 ml sample was placed into a conical centrifuge test tube and was subjected to a G force of 3000 for 1 minute. The results were as follows:

3% oil
16% floating solids “rag”
51% water extract
30% solids

Much better separation of phases was obtained with higher yields of the water extract, which comprises the avocado sugars, including mannoheptulose. This sample subsequently was subjected to centrifugation at 10,000 G but gained only 4% extra settled solids during this “polishing” step.

Results of these experiments showed that (1) samples diluted at a ratio of 2:1 (2 parts water:1 part avocado) and (2) samples diluted at a ratio of 1:1 with acidification yielded a greater volume of water extract after centrifugation compared to (3) samples diluted at a ratio of 1:1 with no acidification. These experiments also demonstrate that that, instead of acidification, emulsification with a food grade emulsifier may increase yield.

Results from these experiments also showed that a yield of about 10% rag (floating solids) is associated with an effective use of a tricanter on 3000 G. With the particular tricanter used in these experiments, 3000 G was the minimum force that achieved effective separation of the water extract comprising carbohydrates from ground avocados. Requisite centrifugal force, in some aspects, depends upon the type of centrifuge used.

Results from these experiments also showed separation of the aqueous phase occurred when the emulsion was heated to around 160 degrees Fahrenheit (71.11° C.).

### Example 4

### Determining Effect of Temperature on Total Cumulative Losses in the Tricanter Process of Preparing Avocado Extracts

Experiments were carried out to determine the effect of temperature on total cumulative losses of mannoheptulose during the extraction process.

Avocados were processed to obtain mannoheptulose through a water extraction. In the extraction process to produce a mannoheptulose sugar concentrate, a tricanter was used to separate oil, solids, and water extract which contains mannoheptulose. Ripened and unripened avocados were evaluated at various temperatures and times in feed tanks prior to the introduction of the tricanter. Feed rates, G force, and equipment parameters were optimized to produce the results of a clarified water extract containing mannoheptulose, separated from oil, and a solids cake material.

Three different temperature conditions (ambient (or room temperature), spiked at 60° C., and continuously heated at 60° C.) were tested on the following types of materials:

(1) emulsified, unripe, frozen, whole Mexican Hass avocado mixed 3 to 1 by weight with water; and (2) emulsified, ripe, frozen Mexican Hass avocado flesh mixed 3 to 1 by weight with water.

It was determined that total cumulative losses of mannoheptulose were lowest when the temperature was spiked. Total cumulative losses were about 59.5% at ambient temperature, 22.0% when spiked at 60 degrees C., and 40.0% when continuous at 60 degrees C.

These experiments showed that grind size, temperature, form of avocado (whole or flesh only), centrifugal force, dilution ratio, and storage times are all important in the tricanter process. A food grade emulsifier effectively prepares the raw material into an emulsion so that effective separation of oil, solids, and water occurs. Temperature of at least 160° F. (about 71.11° C.) facilitates solubilization of the avocado sugars. Pit and peel from whole avocados limited the temperature operating window as compared to the flesh only fruit. 3000 G was the minimum force needed for the effective separation of mannoheptulose extract from ground avocados. Dilution ratio with water influences the mannoheptulose losses in the solids cake. The water extract comprising mannoheptulose was virtually oil free and contained the highest levels of mannoheptulose as desired.

The disclosure has been described in terms of particular embodiments found or proposed to comprise specific modes for the practice of the methods and compositions of the invention described herein. Various modifications and variations of the described invention will be apparent to those skilled in the art without departing from the scope and spirit of the invention. Although the disclosure provides specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A process for preparing a carbohydrate extract comprising mannoheptulose and/or perseitol, the process comprising separating an aqueous phase from other phases of an avocado emulsion by centrifugation to provide a carbohydrate extract comprising at least about 2% mannoheptulose and/or perseitol.

2. The process according to claim 1, wherein carbohydrates in the avocado emulsion are solubilized in the aqueous phase of the emulsion by heating.

3. The process according to claim 1, wherein the avocado emulsion is further combined with an enzyme and/or an acid.

4. The process according to claim 1, further comprising the step of concentrating the carbohydrate extract comprising mannoheptulose utilizing at least one concentration method selected from the group consisting of heating, vacuum drying, evaporating, refractance window drying, freeze drying, and spray drying.

5. The process according to claim 1, wherein centrifugation is carried out by a horizontal or vertical bowl centrifuge.

6. The process according to claim 5, wherein centrifugation is carried out by a two-phase separator or a three-phase separator.

7. The process according to claim 1, wherein the centrifugation is carried out with a relative centrifugal force (G force) of at least about 2000 G.

8. The process according to claim 1, wherein the centrifugation is carried out for at least about 10 minutes.

9. The process according to claim 1, wherein the pH of the avocado emulsion is reduced to a pH of less than about 4.0.

10. The process according to claim 1, wherein the water to avocado ratio in the avocado emulsion is at least about 2:1.

11. The process according to claim 2, wherein the heating is carried out at a temperature from about 20° C. to about 100° C.

12. The process according to claim 2, wherein the avocado emulsion or aqueous phase of the avocado emulsion is heated to at least about 50° C.

13. The process according to claim 2, wherein the heating of the avocado emulsion or aqueous phase of the avocado emulsion is carried out at a target temperature for at least about 30 seconds.

14. The process according to claim 1, wherein the avocado emulsion is prepared from whole fruit or flesh only fruit.

15. The process according to claim 1, wherein yield of mannoheptulose and/or perseitol in the carbohydrate extract is at least about 2 g mannoheptulose and/or perseitol per kg avocado or about 0.2%.

16. A carbohydrate extract prepared according to the process of claim 1.

17. The carbohydrate extract of claim 17 further comprising one or more food composition components.

18. The carbohydrate extract of claim 18, wherein the food composition is a pet food composition.

19. A process for preparing a carbohydrate extract comprising mannoheptulose and/or perseitol, the process comprising the step of:
separating water-soluble components from an avocado emulsion by centrifugation to provide a carbohydrate extract comprising at least about 2% mannoheptulose and/or perseitol; and
filtering the water-soluble components by ultrafiltration.

20. The process of claim 19, further comprising the steps of
(a) heating the aqueous avocado emulsion prior to centrifugation;
(b) separating water-soluble components from the avocado emulsion by centrifugation; and
(c) filtering the water-soluble components by ultrafiltration.
